(19) United States
(12) Patent Application Publication
(10) Pub. No.: US 2017/0007711 A1
(43) Pub. Date: Jan. 12, 2017

(54) PEGYLATED GROWTH HORMONE ANTAGONISTS

(71) Applicant: Richard S. BRODY, Columbus, OH (US)

(72) Inventor: Richard S. BRODY, Columbus, OH (US)

(21) Appl. No.: 15/204,425

(22) Filed: Jul. 7, 2016

Related U.S. Application Data
(60) Provisional application No. 62/189,525, filed on Jul. 7, 2015.

(51) Int. Cl. A61K 47/48; A61K 38/27

(52) U.S. Cl. CPC A61K 47/48215; A61K 38/27

(57) ABSTRACT

A composition that is a human growth hormone receptor antagonist comprising human growth hormone receptor antagonist G120K, wherein at least one amino acid of human growth hormone receptor antagonist G120K has been mutated to cysteine; and a polytethylene glycol molecule (PEG) conjugated to each cysteine residue in the human growth hormone receptor antagonist G120K mutant.

### CROSS-REFERENCE TO RELATED APPLICATIONS

This patent application claims the benefit of U.S. Provisional Patent Application Ser. No. 62/189,525 filed on Jul. 7, 2015 and entitled “Pegylated Growth Hormone Antagonists”, the disclosure of which is hereby incorporated by reference herein in its entirety and made part of the present U.S. utility patent application for all purposes.

### BACKGROUND OF THE INVENTION

The described invention relates in general to compositions for use as receptor antagonists, and more specifically to novel human growth hormone antagonists that have the potential to be highly effective therapeutics.

Human growth hormone, also known as somatotropin or somatropin, is a peptide hormone that stimulates growth, cell reproduction, and regeneration in humans and other animals. Growth hormone is a type of mitogen that is specific only to certain kinds of cells and is a 191-amino acid, single-chain polypeptide that is synthesized, stored, and secreted by somatotropic cells within the lateral wings of the anterior pituitary gland. Acromegaly is a syndrome that results when the anterior pituitary gland produces excess growth hormone (hGH) after epiphyseal plate closure at puberty. If hGH is produced in excess prior to epiphyseal plate closure, the result is gigantism (or giantism). A number of disorders may increase the pituitary's hGH output, although most commonly it involves a tumor called pituitary adenoma, derived from a distinct type of cell (somatotrophs). Acromegaly most commonly affects adults in middle age and can result in severe disfigurement, complicating conditions, and premature death if untreated. Because of its pathogenesis and slow progression, the disease is hard to diagnose in the early stages and is frequently missed for years until changes in external features, especially of the face, become noticeable.

A receptor is a protein molecule usually found embedded within the plasma membrane surface of a cell that receives chemical signals from outside the cell. When such chemical signals bind to a receptor, they cause some form of cellular/tissue response such as, for example, a change in the electrical activity of the cell. In this sense, a receptor is a protein molecule that recognizes and responds to endogenous chemical signals. An agonist, such as human growth hormone, is a chemical composition that binds to a receptor and activates the receptor to produce a biological response. Whereas an agonist causes an action, an antagonist blocks the action of the agonist and an inverse agonist causes an action opposite to that of the agonist. A receptor antagonist is a type of receptor ligand or drug that blocks or dampens agonist-mediated responses rather than provoking a biological response itself upon binding to a receptor. These compositions are sometimes called blockers and examples include alpha blockers, beta blockers, and calcium channel blockers. In pharmacology, antagonists have affinity but no efficacy for their cognate receptors, and binding will disrupt the interaction and inhibit the function of an agonist or inverse agonist at receptors. Antagonists mediate their effects by binding to the active (orthosteric) site or to other (allosteric) sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of drug antagonists achieve their potency by competing with endogenous ligands or substrates at structurally defined binding sites on receptors. By definition, antagonists display no efficacy to activate the receptors they bind and antagonists do not maintain the ability to activate a receptor. Once bound, however, antagonists inhibit the function of agonists, inverse agonists, and partial agonists.

Growth hormone receptor antagonists such as pegvisomant (marketed as Somavert®) are used in the treatment of acromegaly. Such compositions are used if the tumor of the pituitary gland causing the acromegaly cannot be controlled with surgery or radiation and the use of somatostatin analogues is unsuccessful. Pegvisomant is typically delivered as a powder that is mixed with water and injected under the skin.

PEGylation is the process of both covalent and non-covalent amalgamation of polyethylene glycol (PEG) polymer chains to molecules and macrostructures, such as drugs, peptides, antibody fragments, or therapeutic proteins. PEGylation is routinely achieved by incubation of a reactive derivative of PEG with the target molecule and produces alterations in physiochemical properties, including changes in molecular size and molecular charge. These physical and chemical changes increase systemic retention of the therapeutic agent and can influence the binding affinity of the therapeutic moiety to the cell receptors and can alter the absorption and distribution patterns. The covalent attachment of PEG to a drug or therapeutic protein can also “mask” the agent from the host's immune system (i.e., reducing immunogenicity and antigenicity), and increase the hydrodynamic size (i.e., size in solution) of the agent which prolongs its circulatory time by reducing renal clearance. PEGylation can also provide water solubility to hydrophobic drugs and proteins.

PEGylation, by increasing the molecular weight of a molecule, can impart several significant pharmacological advantages over the unmodified form of the molecule, such as: (i) improved drug solubility; (ii) reduced dosage frequency, without diminished efficacy and with potentially reduced toxicity; (iii) extended circulating life; (iv) increased drug stability; and (v) enhanced protection from proteolytic degradation. PEGylated drugs also include the following commercial advantages: (i) opportunities for new delivery formats and dosing regimens; and (ii) extended patent life of previously approved drugs. PEG is a particularly attractive polymer for conjugation and the specific characteristics of PEG moieties relevant to pharmaceutical applications include: (i) water solubility; (ii) high mobility in solution; (iii) lack of toxicity and low immunogenicity; and (v) altered distribution in the body.

The addition of high molecular weight polyethylene glycols (PEGs) to proteins has been previously shown to increase the in-vivo half-lives of these proteins by a size dependent decrease in elimination by the kidneys. The addition of PEGs also lowers the immunogenicity of the proteins and decreases aggregation and protease cleavage (Pasut and Vronese, 2012; and Parveen and Sahoo, 2006). Multiple known PEGylated proteins have been approved by the USFDA for therapeutic use, including hormones, cytokines, antibody fragments, and enzymes (Pasut, and Veronese, 2012; Alconcel et al., 2011; and Kling, 2013). Thus, there is an ongoing need for the further development of PEGylated therapeutics, particularly for use in the treatment of diseases that are responsive to the use of human growth hormone (hGH) receptor antagonists or other receptor antagonists.

### SUMMARY OF THE INVENTION

The following provides a summary of certain exemplary embodiments of the present invention. This summary is not an extensive overview and is not intended to identify key or critical aspects or elements of the present invention or to delineate its scope. As will be appreciated by one skilled in the art, the single letter amino acid abbreviations used herein follow the IUPAC format.

In accordance with one aspect of the present invention, a first composition or compound that functions as a human growth hormone receptor antagonist is provided. This human growth hormone receptor antagonist includes human growth hormone receptor antagonist G120K, wherein at least one amino acid of human growth hormone receptor antagonist G120K has been mutated to cysteine; and a polytethylene glycol molecule conjugated to each cysteine residue in the human growth hormone receptor antagonist G120K mutant.

In accordance with another aspect of the present invention, a second composition or compound that functions as a human growth hormone receptor antagonist is provided. This human growth hormone receptor antagonist includes human growth hormone receptor antagonist G120K, wherein at least one amino acid of human growth hormone receptor antagonist G120K has been mutated to cysteine, and wherein the amino acid mutated to cysteine is T3, E39, P48, Q69, N99, G120, T142, H151 or combinations thereof; and a polytethylene glycol molecule conjugated to each cysteine residue in the human growth hormone receptor antagonist G120K mutant, wherein the polytethylene glycol molecule is a linear structure or a branched structure.

In yet another aspect of this invention, a third composition or compound that functions as a human growth hormone receptor antagonist is provided. This human growth hormone receptor antagonist includes human growth hormone receptor antagonist G120K, wherein at least one amino acid of human growth hormone receptor antagonist G120K has been mutated to cysteine, and wherein the amino acid mutated to cysteine is T3, E39, P48, Q69, N99, G120, T142, H151 or combinations thereof; and a polytethylene glycol molecule conjugated to each cysteine residue in the human growth hormone receptor antagonist G120K mutant, wherein the polytethylene glycol molecule is a linear structure or a branched structure, wherein the polyethylene glycol molecule contains a malemide group for conjugation to a free sulfhydryl group, and wherein the polyethylene glycol molecule is selected from the group consisting of a 20 kDa linear molecule, a 40 kDa linear molecule, and a 40 kDa branched molecule.

Additional features and aspects of the present invention will become apparent to those of ordinary skill in the art upon reading and understanding the following detailed description of the exemplary embodiments. As will be appreciated by the skilled artisan, further embodiments of the invention are possible without departing from the scope and spirit of the invention.

### DETAILED DESCRIPTION OF THE INVENTION

Exemplary embodiments of the present invention are described below. Although the following detailed description contains many specifics for purposes of illustration, a person of ordinary skill in the art will appreciate that many variations and alterations to the following details are within the scope of the invention. Accordingly, the following embodiments of the invention are set forth without any loss of generality to, and without imposing limitations upon, the claimed invention.

The present invention provides novel human growth hormone (hGH) antagonists for use primarily as therapeutics. The hGH antagonists of this invention are typically made by mutating one or more selected amino acids of hGH G120K, a known hGH antagonist, to cysteines and then conjugating the cysteines to chemically activated polyethylene glycol molecules. The positions of the substituted cysteines have been selected for minimal loss in receptor binding activity after conjugation with polyethylene glycol. The specific type and number of polyethylene glycol modifiers of this invention have been selected to produce antagonists with increased in-vivo half-lives.

Two important variables in the preparation of PEGylated proteins in accordance with this invention are: (i) the amino acid position used for PEG attachment; and (ii) the size and type of the conjugated PEG. Initial research with similar compositions was done using random attachment of relatively small PEGs (e.g., about 5 kDa) to multiple lysines on the surfaces of proteins. This procedure successfully increased the in vivo half-lives of the proteins, but resulted in large decreases in the affinity of the proteins for their receptors. More recent experimental approaches have added PEG molecules to specific amino acid sites on proteins. Two common methods used for site specific PEGylation are: (i) addition of PEG to the N-terminal amine of proteins by way of low pH reductive amination; and (ii) addition of PEG to the thiol groups of cysteines that are either native to the protein or engineered into specific positions. Other methods include PEG addition to unnatural amino acids; PEG addition to proteins C-termini by way of intein fusion proteins; and PEG addition to accessible glutamines by way of transaminase catalysis (Pasut and Veronese, 2012).

Two different types of polyethylene glycol (PEG) molecules are utilized with the present invention. The first type of PEG is prepared by polymerization and is by nature polydispersed, in that there is a distribution of molecular weight products around the average molecular weight. The second class of polyethylene glycols are discrete PEGs (dPEG®s; Quanta BioDesign). Such dPEG®s are single PEG molecules that are prepared by step-wise, organic chemistry so that each dPEG® species is a pure single compound with a specific structure and molecular weight (Povosky et al., 2013). The different types of PEGs have been produced as both linear and branched structures. For large polydispersed PEGs, the addition of a branched PEG to a protein may cause less of a decrease in binding affinity and a greater increase in half-life than addition of a linear PEG of the same molecular weight (Zhang et al., 2012). Branched dPEG®s have been shown to increase protein half-lives and a negatively charged dPEG® has been shown to be particularly efficacious (Ding et al., 2013).

PEGylated Growth Hormone Antagonists

The conversion of hGH from a growth agonist to a growth antagonist requires only a single amino acid change at hGH position 120 from the native glycine to any amino acid except alanine (Chen et al., 1994). This molecule, however, cannot be used as a therapeutic for conditions of excess growth (e.g., acromegaly) due to its short in vivo half-life. Researchers have addressed this problem by the addition of polyethylene glycol molecules to the hGH antagonist hGH G120K to decrease the clearance of the molecule through the kidneys. Somavert®, an FDA approved treatment for acromegaly, contains 4-6 linear PEG molecules with molecular weights of 5000 Daltons each. The addition of the PEGs, which are attached randomly to surface lysines (van der Lely and Kopchick, 2006), increases the in vivo half-life of the antagonist from less than an hour to approximately 72 hours (Finn, 2009). The affinity of the PEGylated antagonist for the membrane bound receptor, however, is reduced approximately 30 fold compared with the unpegylated molecule (Ross et al., 2001). Despite the decrease in receptor affinity, Somavert® is an effective treatment for acromegaly, although a large daily dose of 5-30 mg is typically prescribed.

The loss of receptor binding that occurs after the addition of multiple low molecular weight PEGs (about 5 kDa) to random lysines on a protein was common with early protein-PEG conjugates (Parveen and Sahoo, 2006). More recently, however, researchers have made conjugates with higher receptor binding activity by adding a single higher molecular weight PEG to specific amino acid positions on the protein target (Pasut and Veronese, 2012). In the case of growth hormone antagonists, either a 20 kDa or a 40 kDa linear PEG was added to the N-terminus of the unpegylated precursor to Somavert® (B2036) using reductive alkylation at a low pH (Wu et al., 2013). The addition of the 20 kDa PEG and the 40 kDa PEG reduced the affinity of the antagonist for the soluble hGH receptor by about 50% and about 95%, respectively. The ability of these molecules to inhibit the production of insulin growth factor-1 (IGF-1) in rats was tested. While the 40 kDa PEG conjugate was inactive, the 20 kDa conjugate reduced the IGF-1 production by 30 to 40%.

Pegylated Growth Hormone

Insights regarding the predicted effects of different PEGylation strategies on growth hormone antagonist activity and half-life can be obtained by analysis of the results from PEGylation of human growth hormone (hGH) (Finn, 2009). Cox and coworkers (2007) mutated the threonine at position 3 of human growth hormone to cysteine (T3C hGH) and then conjugated the cysteine to a 20 kDa linear PEG. The activity of hGH, measured by a proliferation assay, decreased by about 4 fold and the half-life increased by about 8 fold. Similar increases in half-life occurred after the enzyme catalyzed addition of a 20 kDa PEG to Gln141 and the chemical addition of a 20 kDa PEG to the N-terminus (Freitas et al., 2013). The addition of a 30 kDa linear PEG to different amino acid positions, which were mutated to chemically active non-native amino acids, resulted, in the optimal cases, in a roughly 10 fold loss of proliferation activity and a roughly 10 fold increase in half-life (Cho et al., 2011). The addition of a linear 43 kDa PEG to position 141 (Gln mutated to cysteine) was reported to increase the half-life ˜30 fold and the addition of a branched 40 kDa PEG to the N-terminus of hGH was reported to increase the half-life about 20 fold (Rasmussen et al., 2010).

PEGylated Cytokines

The site specific addition of PEGs to cytokines, which have molecular weights similar to that of hGH, indicates that a single PEG can cause a significant increase in half-life. Rosendahl et al. (2005) reported that addition of a 10 kDa PEG, a 20 kDa PEG, and a 40 kDa PEG to position 5 of Interferon α-2, after mutation of this position to cysteine, reduced the in vitro bioactivity by a factor of 2-3. In contrast, the half-life increased for the 10 kDa, 20 kDa PEG, and 40 kDa PEG by factors of 14, 23 and 40 respectively. A similar result was found for the addition of different molecular weight PEGs to human granulocyte-macrophage colony-stimulating factor (Doherty et al., 2005). Bell et al. (2008) found that the addition of either a 20 kDa or a 40 kDa PEG to position 111 (M111C) of interferon α led to a three-fold decrease in receptor binding activity. The half-lives for the 20 kDa and the 40 kDa substituted interferons increased by 25 fold and 39 fold respectively. In contrast, the site specific addition of a 40 kDa PEG to interferon β-1b only increased the half-life of by about three-fold (Lee et al., 2013).

Qiu et al. (2013) substituted position 22 of human thyroid stimulating hormone with a linear 40 kDa PEG, a two branched 40 kDa PEG, and a three branched 40 kDa PEG and found that the receptor binding activity decreased by 5 fold, 14 fold, and 11 fold respectively. A comparison of the half-lives of these different conjugates was not reported. Fam et al. (2014) studied the effect of the addition of 10 kDa, 20 kDa, and 40 kDa PEGs to position 103 of interferon γ and found that the PEGs did not significantly affect the cytokine's in vitro activity, but in all cases the half-life was increased by about 30 fold.

Antibody Fragments

Extensive work has been done on the addition of PEGs to antibody fragments to increase their residence time in the body. Lee et al. (1999) conjugated a single chain antibody fragment (scFv MAb; 26 kDa) with six different PEG polymers with MWs ranging from 2 to 20 kDa. These conjugates showed longer half-lives compared to their nonPEGylated parent. Increasing PEG polymer length was found to be more effective for half-life extension than increasing total PEG mass. Li et. al. (2010) showed that the addition of two discrete linear PEG units conjugated to random lysines on a diabody resulted in longer blood retention times than unpegylated or polydispersed PEGylated products. Chapman et al. (2002) demonstrated that the half-lives of antibody Fab′ fragments (about 50 kDa) are directly related to the size and numbers of site-directed PEGs.

Lee et al. (2013) found that an increase in linear, randomly conjugated PEG mass (4-20 kDa) in scFv-PEG conjugates effectively increased half-lives roughly linearly with mass. These workers found that a single 20 kDa PEG was more effective than four 5,000 PEGs, concluding that PEG length was more important than PEG mass. In a study of the roughly 50 kDa Fab′ antibody fragment, the site specific addition of a 4.4 kDa branched discrete PEG (dPEG) increased the half-life by a factor of about two over unconjugated Fab′ (Ding et al., 2013).

Selection of Amino Acid Positions of hGH G120K to Mutate to Cysteine

In various embodiments of the present invention, PEGylated versions of the antagonist hGH G120K were prepared by attaching PEGs to cysteine residues that have been incorporated into the antagonist sequence through genetic engineering. The antagonist positions selected for mutation to cysteine were selected using the X-ray structure of the complex of hGH with an hGH receptor dimer (hGHR2; Sundstrom et al., 1996). The structure of hGH when bound to hGHR2 is almost identical to the structure of the hGH antagonist hGH G120R when the antagonist is bound to the same receptor (Sundstrom et al., 1996).

Two criteria, based on the hGH-hGHR2 crystal structure, were used to select amino acids for cysteine mutation: (i) accessibility of the amino acid to solvent; and (ii) making the substitution of cysteine for the selected amino acid needs close to an energetically neutral process. The solvent accessibility of each amino acid in hGH-hGHR2 was determined by way of the modeling programs Swiss PDB Viewer (Guex and Peitsch, 1997) and PoPMuSIC (Dehouck et al., 2009; Dehouck et al., 2011). The energetic cost of substituting cysteine for each amino acid position was determined using the Prediction of Protein Mutant Stability Changes (PoPMuSIC) program (http://babylone.ulb.ac.be/popmusic/).

The amino acids selected by solvent (e.g., water) accessibility and mutation energy considerations are listed below in Table 1 under “All Selected Positions” in seven spatially separate domains. However, being accessible to water is not necessarily a sole criteria for selection; the amino acids need to be accessible to the much larger PEG molecules in order for the PEGylated antagonists to bind to a target receptor. The X-ray structure of hGH-hGHR2 was inspected to determine if the side chains of the selected amino acids are directed towards solvent or towards the hGH-hGHR2 protein complex. Amino acid positions whose side chains point into the solvent are the most desirable candidates for PEG substitution and are listed below in Table 1 under “Final Selection”. An additional position, H151, was also selected for cysteine mutation. This position is part of a section of Loop 3 that was not apparent in the crystal structure. The general location of the missing segment is pointing away from the hGH-hGHR2 protein complex.

TABLE 1
Final Selection of Amino Acid Positions

| | | Final Selection |
|---|---|---|
| | | (amino acids whose side |
| | All Selected | chains point away from |
| | Positions | the protein structure) |
| Domain 1 (N-Terminus): | F1, T3 | T3 |
| Domain 2 (Loop 1): | E39 | E39 |
| Domain 3 (Loop 1): | P48 | P48 |
| Domain 4 (Loop 1): | Q69 | Q69 |
| Domain 5 (Loop 2): | N99, L101 | N99, L101 |
| Domain 6 (Loop 3): | T142, D147, D154 | T142 |
| Domain 7 (C-Terminus) | G190 | G190 |

Selection of PEGs for Conjugation to hGH G120K Mutants

Two different classes of polyethylene glycol (PEG) molecules are utilized with the present invention. The first class of PEGs was prepared by polymerization and has been used to modify proteins in order to increase their in vivo half-lives (Kling, 2013). This type of PEG is by nature polydispersed, meaning that there is a distribution of molecular weight products around the average molecular weight. The PEGs include a 20 kDa linear PEG (Layson Bio, MPEG-MAL-20,000), a 40 kDa branched PEG (NOF, Sunbright GL2-400MA), and a linear 40 kDa PEG (NOF, Sunbright ME-400MA). These PEGS each contain a malemide group for conjugation to the free sulfhydryl groups of the mutant proteins. The second class of polyethylene glycols are “discrete” PEGs (dPEG®s; Quanta BioDesign). These dPEG®s are pure single PEG molecules that are prepared using step-wise, organic chemistry so that each dPEG® species is a pure single compound with a specific structure and molecular weight (Povosky et al., 2013). The dPEGs used in this invention, which typically contain a maleimide group for coupling to free thiols, include the following: a tri-branched molecule with a molecular weight of 4473 Daltons and a carboxylate anion at the terminus of each branch (Quanta BioDesign #10451, MAL-dPEGA); a neutral tri-branched molecule with a molecular weight of 4299 Daltons (Quanta BioDesign #4229, MAL-dPEGB); a neutral 9-branched molecule with a molecular weight of 8324 (Quanta Biodesign #10484; MAL-dPEGE); and a neutral 9-branched molecule with a molecular weight of 15,592 (Quanta Biodesign #11487; MAL-dPEGF). The tri-branched 4473 Da molecule has been conjugated to an antibody fragment and its effect on blood clearance in mice has been investigated (Ding et al., 2013). While the added dPEG® increased the molecular weight of a 50 kDa protein molecular weight by only about 8%, the “area under the curve” (AUC) for blood clearance increased by a factor of about 2.5 over the AUC for the unPEGylated protein.

Purification and Pegylation of hGH G120K Mutants

Cell Disruption

A cell pellet obtained from centrifugation of 250 mL of growth medium containing the expressed mutant was suspended in 10 mL PBS and combined with 0.05 mL of a protease inhibitor cocktail without EDTA (Sigma P8849). The solution was cooled in an ice water mixture and sonicated for four minutes in 30 second bursts. After each sonication, the sample was cooled in the ice-water mixture until the temperature was below 4° C. The sonicated suspension was then centrifuged at 4° C. and 25,000×g for 30 minutes and the supernatant was collected and kept on ice.

Affinity Purification

The sonicated supernatant was adjusted to 0.3 M sodium chloride and made 5 mM imidazole by addition of a pH 7 solution of 150 mM imidazole. The sample was then applied to a gravity flow column having a stoppered outlet packed with 5 mL of TALON® (Clontech) immobilized metal affinity resin (IMAC). The resin was equilibrated in 0.05 M sodium phosphate buffer, pH 7.0 containing 5 mM imidazole and 0.3 M sodium chloride prior to addition of the supernatant. The top of the column was then also stoppered and the column mixed end-over-end at room temperature for 30 minutes. The column was then allowed to drain and washed with at least five 5 mL aliquots of equilibration buffer. Washing was continued until the A(280) nm of the eluent no longer decreased. The column was then eluted with pH 7 equilibration buffer containing 150 mM imidazole and the product containing fractions were made 5 mM EDTA by addition of a 100 mM solution of disodium EDTA adjusted to pH 7.

TEV Protease Cleavage of His-Tag

The IMAC purified mutant was concentrated by molecular filtration to 2 mg/mL and 0.5 mL of the solution was combined with 0.05 mL of a solution containing 15 mM reduced glutathione+1.5 mM oxidized glutathione. An aliquot of 0.04 mg TEV Protease (TurboTev, Accelagen) was then added and the solution incubated for two hours at room temperature followed by overnight incubation at 4° C. The imidazole containing buffer was then exchanged on a spin column for a buffer containing 0.05 M sodium phosphate, pH 7.0 and 0.3 M sodium chloride.

Pegylation and Purification

The desalted mutant was PEGylated by making the solution 0.5 mM maleimide-PEG and incubating the reaction for two hours at room temperature followed by overnight incubation at 4° C. The PEGylated mutant was then applied to a gravity flow IMAC column containing 1 mL TALON® resin equilibrated in the spin column buffer and the column was washed with 5 CVs of the same buffer. The TALON® flow through and wash contained the product, which was then concentrated by a centrifugal concentrator to 0.3 mL and purified by size exclusion chromatography on a Superdex 200 Increase 10/300 GL column (GE Healthcare) equilibrated in 0.05 M Tris Buffer, pH 8, containing 0.15 M sodium chloride and 10% glycerol. The product fractions were combined and analyzed for protein concentration by absorption at A(280) nm and for purity by SDS-PAGE. The addition of a single dPEGB to the single cysteine mutants and two dPEGBs to the double cysteine mutants was confirmed by MALDI mass spectrometry.

Competition ELISA Assay of Relative Affinity for the hGH Receptor

A Competition ELISA required the preparation of biotinylated hGH, which was prepared by standard methods (Hermanson, 2008) using Biotin-dPEG12-NHS (Quanta BioDesign). Microtiter plates (Corning 96 well plates, half-area, polystyrene) were coated with 0.05 mL of 0.125 μg/mL solutions of the hGH receptor (R&D Systems, 1210-GR-50; cloned as a chimira with an antibody Fc region) in a 0.05 M sodium carbonate buffer at pH 9.6 and incubated either at 37° C. for one hour or overnight at 4° C. After washing the plate three times, with three minute incubations between washes, with of 0.125 mL PBS, 0.05% Tween 20 (Wash Buffer), the plates were blocked for one hour by incubation with 2% BSA in PBS.

Preliminary ELISA assays were performed to determine the concentration of biotin-hGH to use in the completion ELISA. Plates coated with the hGH receptor and blocked were incubated for one hour at RT with different concentrations of biotinylated hGH dissolved in PBS, 0.1% BSA, 0.05% Tween 20 (Dilution Buffer). The plates were then washed 3× with Wash Buffer and incubated for 1 hour at room temperature with 0.5 ug/mL Streptavidin-HRP (Pierce, 21130) in Dilution Buffer. The plates were again washed 3 times and developed by the addition of 0.05 mL TMB (KPL). After incubation for 2-20 minutes at room temperature, the plates were then quenched by the addition of 0.1 mL 1 M HCl and read on a plate reader at 450 nm.

A concentration of biotin-hGH was selected such that the assay response was in the linear range of the plot of biotin-hGH versus A(450) nm (approximately 1 OD unit). Competition assays were performed by the preparation of solutions in polypropylene 96 well plates that contained the selected concentration of biotin-hGH and varying concentrations of hGH, hGH G120K mutant, or PEGylated hGH G120K mutant. Ninety-six well immunoassay plates were coated with the hHG receptor, blocked as described above, and then incubated for 1 hour at RT with the solutions containing the selected concentration of biotin-hGH and different concentrations of the inhibitors. The plates were then washed and treated with Streptavidin-HRP and TMB as described above.

The concentration of recombinant hGH that gave a 50% inhibition of the assay response (IC₅₀) was used as the standard to determine the relative affinities of the mutants for the hGH receptor. Each assay plate contained a series of concentrations of both the hGH standard and the mutants to be tested and the relative 1050 values were determined. Two polydispersed PEGs (ME400MA and GL2-400MA) were conjugated to the free thiols of hGH G120K-H151C and hGH G120K-N99C. G120K-H151C-ME400MA and G120K-H151C-GL2-400MA had, respectively, 20% and 50% of the inhibitory activity of G120K. N99C-ME400MA and N99C-GL2-400MA had 20% and 2% of the inhibitory activity of G120K.

The binding affinities of the different dPEG® conjugated mutants of the present invention relative to that of hGH are shown in Table 2, below. Seven single mutants and three double mutants were conjugated to a single tri-branched molecule dPEG® with a molecular weight of 4473 Daltons (dPEGA) and the molecule was purified as described. As shown in Table 2, certain of these single mutants were also conjugated to three other dPEGs. Three double cysteine mutants were also prepared and conjugated to different dPEGs, as shown in Table 2.

TABLE 2
Receptor Binding Activity of hGH 120K Mutants ¹

| | Percent Receptor Binding Activities | | | |
|---|---|---|---|---|
| | Relative to that of hGH Determined | | | |
| hGH Mutant -All Mutants | from the Concentration of Each Sample | | | |
| Contain the G120K Mutation | that Yields 50% Inhibition (I₅₀) | | | |
| dPEG Substitution | dPEGA² | dPEGB² | dPEGE² | dPEGF² |
| G120K-T3C-dPEGX | 70 | 70 | NT | NT |
| G120K-E39C-dPEGX | 20 | NT | NT | NT |
| G120K-P48C-dPEGX | 20 | NT | NT | NT |
| G120K-Q69C-dPEGX | 20 | NT | NT | NT |
| G120K-N99C-dPEGX | 90 | 70 | 40 | 4 |
| G120K-T142C-dPEGX | 50 | 90 | 50 | 20 |
| G120K-H151C-dPEGX | 100 | 60 | 40 | 4 |
| G120K-N99C-dPEGX- | 20 | 40 | 20 | —³ |
| H151C-dPEGX | | | | |
| G120K-T142C-dPEGX- | 50 | 80 | 30 | —³ |
| N99C-dPEGX | | | | |
| G120K-T142C-dPEGX- | 50 | 40 | 10 | —³ |
| H151C-dPEGX | | | | |

¹ The receptor binding activities were determined using a competitive ELISA where the recombinant receptor was bound to a plate and the concentration of each sample needed to inhibit the binding of biotin-hGH to the coated plate by 50% (I50) was determined. The Table entries show the I₅₀s relative to that of hGH, which is defined as 100%, and are rounded to a single significant figure. Only a single competitive ELISA was run for most of the mutants and the estimated relative standard deviation is 25%. Entries marked NT were not tested in this assay.
²dPEGA is a tri-branched molecule with a molecular weight of 4473 Daltons and a carboxylate anion at the terminus of each branch; dPEGB is a neutral tri-branched molecule with a molecular weight of 4299 Daltons, dPEGE is a neutral 9-branched molecule with a molecular weight of 8324; and dPEGF is a neutral 9-branched molecule with a molecular weight of 15,592.
³These reactions did not proceed to the double PEGylated product.

Western Blot Assay for the Ability of PEGylated Mutants to Inhibit the Stimulation of STAT 5 Phosphorylation by hGH

The ability of the PEGylated mutants of the present invention to inhibit the stimulation of Stat5 Protein phosphorylation by hGH was measured in a cell based assay. IM9 cells were incubated in RPMI media for two hours. The cells were then resuspended in fresh RPMI media at 1 million cells per mL and treated with either hGH, PEGylated hGH mutants, or hGH+PEGylated mutants at concentrations from 0 to 5000 ng/mL. The treated cells were then incubated for 15 minutes at 37° C. in a 5% carbon dioxide incubator. The cells were then spun down, lysed in a buffer containing 1% Triton X-100 and sodium orthovanadate, and loaded on an SDS PAGE gel. The gel was run under standard conditions and the proteins then transferred electrophoretically to a PVDF membrane. The membrane was blocked and then incubated overnight at 4° C. with a mixture of rabbit anti-Stat5 Protein antibody and rabbit anti-β-actin antibody (positive cell control). The membrane was then washed and incubated with a HRP conjugated goat anti-rabbit antibody for one hour at room temperature. Finally, the bands were visualized using Pierce Supersignal West chemiluminescent substrate. Qualitative results for the PEGylated mutants are given in Table 3, below.

TABLE 3
Western Blot Assay for the Ability of the PEGylated Mutants
to Inhibit the Stimulation of STAT 5 Phosphorylation by hGH¹

| | Ability of the Pegylated Mutants to | | | |
|---|---|---|---|---|
| hGH Mutant -All Mutants | Inhibit the Stimulation of STAT 5 | | | |
| Contain the G120K Mutation | Phosphorylation by hGH | | | |
| dPEG Substitution | dPEGA² | dPEGB² | dPEGE² | dPEGF² |
| G120K-T3C-dPEGX | NT | + | NT | NT |
| G120K-E39C-dPEGX | NT | NT | NT | NT |
| G120K-P48C-dPEGX | NT | NT | NT | NT |
| G120K-Q69C-dPEGX | NT | NT | NT | NT |
| G120K-N99C-dPEGX | + | + | NT | NT |
| G120K-T142C-dPEGX | + | + | + | + |
| G120K-H151C-dPEGX | + | + | NT | NT |
| G120K-N99C-dPEGX- | + | + | NT | NT |
| H151C-dPEGX | | | | |
| G120K-T142C-dPEGX- | + | + | + | NT |
| N99C-dPEGX | | | | |
| G120K-T142C-dPEGX- | NT | + | + | NT |
| H151C-dPEGX | | | | |

¹The Western assay qualitatively measures the abilities of hGH antagonists to inhibit the hGH stimulation of STAT 5 phosphorylation. The inhibition is expressed as relative to the inhibition obtained with the parent antagonist hGH G120K. In all cases, the relative abilities of the pegylated antagonists to inhibit STAT 5 phosphorylation was between ~20% and ~100% that of hGH G120K. The variation between duplicate runs was too great to make this a quantitative assay. Entries marked NT were not tested in this assay.
²dPEGA is a tri-branched molecule with a molecular weight of 4473 Daltons and a carboxylate anion at the terminus of each branch; dPEGB is a neutral tri-branched molecule with a molecular weight of 4299 Daltons, dPEGE is a neutral 9-branched molecule with a molecular weight of 8324; and dPEGF is a neutral 9-branched molecule with a molecular weight of 15,592.

As indicated by the disclosure above, the compositions of the present invention provide novel human growth hormone receptor antagonists that are useful in therapeutic applications. For reference purposes, SEQ ID NO: 1 provides the DNA sequence for human growth hormone WThGH and SEQ ID NO: 2 and provides the amino acid sequence for human growth hormone WThGH. Human growth hormone receptor antagonist G120K is the parent receptor antagonist for the compositions of the present invention, and for reference purposes, SEQ ID NO: 3 provides the DNA sequence for human growth hormone receptor antagonist G120K and SEQ ID NO: 4 provides the amino acid sequence for human growth hormone receptor antagonist G120K. As previously stated, the single letter amino acid abbreviations used herein follow the IUPAC format.

A first human growth hormone antagonist in accordance with an exemplary embodiment of the present invention includes human growth hormone antagonist G120K, wherein amino acid T3 has been mutated to cysteine, and wherein a polytethylene glycol molecule has been conjugated to the cysteine mutation. SEQ ID NO: 5 provides the DNA sequence for human growth hormone antagonist G120K-T3C and SEQ ID NO: 6 provides the amino acid for sequence human growth hormone antagonist G120K-T3C.

A second human growth hormone antagonist in accordance with an exemplary embodiment of the present invention includes human growth hormone antagonist G120K, wherein amino acid E39 has been mutated to cysteine, and wherein a polytethylene glycol molecule has been conjugated to the cysteine mutation. SEQ ID NO: 7 provides the DNA sequence for human growth hormone antagonist G120K-E39C and SEQ ID NO: 8 provides the amino acid sequence for human growth hormone antagonist G120K-E39C.

A third human growth hormone antagonist in accordance with an exemplary embodiment of the present invention includes human growth hormone antagonist G120K, wherein amino acid P48 has been mutated to cysteine, and wherein a polytethylene glycol molecule has been conjugated to the cysteine mutation. SEQ ID NO: 9 provides the DNA sequence for human growth hormone antagonist G120K-P48C and SEQ ID NO: 10 provides the amino acid sequence for human growth hormone antagonist G120K-P48C.

A fourth human growth hormone antagonist in accordance with an exemplary embodiment of the present invention includes human growth hormone antagonist G120K, wherein amino acid Q69 has been mutated to cysteine, and wherein a polytethylene glycol molecule has been conjugated to the cysteine mutation. SEQ ID NO: 11 provides the DNA sequence for human growth hormone antagonist G120K-Q69C and SEQ ID NO: 12 provides the amino acid sequence for human growth hormone antagonist G120K-Q69C.

A fifth human growth hormone antagonist in accordance with an exemplary embodiment of the present invention includes human growth hormone antagonist G120K, wherein amino acid N99 has been mutated to cysteine, and wherein a polytethylene glycol molecule has been conjugated to the cysteine mutation. SEQ ID NO: 13 provides the DNA sequence for human growth hormone antagonist G120K-N99C and SEQ ID NO: 14 provides the amino acid sequence for human growth hormone antagonist G120K-N99C.

A sixth human growth hormone antagonist in accordance with an exemplary embodiment of the present invention includes human growth hormone antagonist G120K, wherein amino acid T142 has been mutated to cysteine, and wherein a polytethylene glycol molecule has been conjugated to the cysteine mutation. SEQ ID NO: 15 provides the DNA sequence for human growth hormone antagonist G120K-T142C and SEQ ID NO: 16 provides the amino acid sequence for human growth hormone antagonist G120K-T142C.

A seventh human growth hormone antagonist in accordance with an exemplary embodiment of the present invention includes human growth hormone antagonist G120K, wherein amino acid H151 has been mutated to cysteine, and wherein a polytethylene glycol molecule has been conjugated to the cysteine mutation. SEQ ID NO: 17 provides the DNA sequence for human growth hormone antagonist G120K-H151C and SEQ ID NO: 18 provides the amino acid sequence for human growth hormone antagonist G120K-H151C.

An eighth human growth hormone antagonist in accordance with an exemplary embodiment of the present invention includes human growth hormone antagonist G120K, wherein amino acids N99 and H151 have been mutated to cysteine, and wherein a polytethylene glycol molecule has been conjugated to each cysteine mutation. SEQ ID NO: 19 provides the DNA sequence for human growth hormone antagonist G120K-N99C-dPEGX-H151C and SEQ ID NO: 20 provides the amino acid sequence for human growth hormone antagonist G120K-N99C-dPEGX-H151C.

A ninth human growth hormone antagonist in accordance with an exemplary embodiment of the present invention includes human growth hormone antagonist G120K, wherein amino acids T142 and N99 have been mutated to cysteine, and wherein a polytethylene glycol molecule has been conjugated to each cysteine mutation. SEQ ID NO: 21 provides the DNA sequence for human growth hormone antagonist G120K-T142C-dPEGX-N99C and SEQ ID NO: 22 provides the amino acid sequence for human growth hormone antagonist G120K-T142C-dPEGX-N99C.

A tenth human growth hormone antagonist in accordance with an exemplary embodiment of the present invention includes human growth hormone antagonist G120K, wherein amino acids T142 and H151 have been mutated to cysteine, and wherein a polytethylene glycol molecule has been conjugated to each cysteine mutation. SEQ ID NO: 23 provides the DNA sequence for human growth hormone antagonist G120K-T142C-dPEGX-H151C and SEQ ID NO: 24 provides the amino acid sequence for human growth hormone antagonist G120K-T142C-dPEGX-H151C.

While the present invention has been illustrated by the description of exemplary embodiments thereof, and while the embodiments have been described in certain detail, there is no intention to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the general inventive concept.

What is claimed:

1) A human growth hormone receptor antagonist, comprising:
(a) human growth hormone receptor antagonist G120K, wherein at least one amino acid of human growth hormone receptor antagonist G120K has been mutated to cysteine; and
(b) a polytethylene glycol molecule conjugated to each cysteine residue in the human growth hormone receptor antagonist G120K mutant.

2) The composition of claim 1, wherein the amino acid mutated to cysteine is T3, E39, P48, Q69, N99, G120, T142, H151 or combinations thereof.

3) The composition of claim 1, wherein the polyethylene glycol molecule is prepared by polymerization and is polydispersed.

4) The composition of claim 1, wherein the polyethylene glycol molecule is a discrete polyethylene glycol that is prepared by step-wise organic chemistry such that each polyethylene glycol species is a substantially pure single compound with a specific structure and molecular weight.

5) The composition of claim 1, wherein the polyethylene glycol molecule is a linear structure or wherein the polyethylene glycol molecule is a branched structure.

6) The composition of claim 1, wherein the polyethylene glycol molecule is selected from the group consisting of a 20 kDa linear polyethylene glycol molecule, a 40 kDa linear polyethylene glycol molecule, and a 40 kDa branched polyethylene glycol molecule.

7) The composition of claim 1, wherein the polyethylene glycol molecule contains a malemide group for conjugation to a free sulfhydryl group.

8) The composition of claim 1, wherein the human growth hormone receptor antagonist is encoded by a DNA sequence having at least 95% identity to a DNA sequence selected from the group consisting of SEQ ID NOs: 5, 7, 9, 11, 13, 15, 17, 19, 21, and 23.

9) The composition of claim 1, wherein the human growth hormone receptor antagonist has an amino acid sequence selected from the group consisting of SEQ ID NOs: 6, 8, 10, 12, 14, 16, 18, 20, 22, and 24.

10) A method for treating a disease or condition responsive to human growth hormone receptor antagonists, comprising administering to the patient an effective amount of the composition of claim 1.

11) A human growth hormone receptor antagonist, comprising:
(a) human growth hormone receptor antagonist G120K, wherein at least one amino acid of human growth hormone receptor antagonist G120K has been mutated to cysteine, and wherein the amino acid mutated to cysteine is T3, E39, P48, Q69, N99, G120, T142, H151 or combinations thereof; and
(b) a polytethylene glycol molecule conjugated to each cysteine residue in the human growth hormone receptor antagonist G120K mutant, wherein the polytethylene glycol molecule is a linear structure or a branched structure.

12) The composition of claim 11, wherein the polyethylene glycol molecule is prepared by polymerization and is polydispersed.

13) The composition of claim 11, wherein the polyethylene glycol molecule is a discrete polyethylene glycol that is prepared by step-wise organic chemistry such that each polyethylene glycol species is a pure single compound with a specific structure and molecular weight.

14) The composition of claim 11, wherein the polyethylene glycol molecule is selected from the group consisting of a 20 kDa linear polyethylene glycol molecule, a 40 kDa linear polyethylene glycol molecule, and a 40 kDa branched polyethylene glycol molecule.

15) The composition of claim 11, wherein the polyethylene glycol molecule contains a malemide group for conjugation to a free sulfhydryl group.

16) The composition of claim 11, wherein the human growth hormone receptor antagonist is encoded by a DNA sequence having at least 95% identity to a DNA sequence selected from the group consisting of SEQ ID NOs: 5, 7, 9, 11, 13, 15, 17, 19, 21, and 23.

17) The composition of claim 11, wherein the human growth hormone receptor antagonist has an amino acid sequence selected from the group consisting of SEQ ID NOs: 6, 8, 10, 12, 14, 16, 18, 20, 22, and 24.

18) A method for treating a disease or condition responsive to human growth hormone receptor antagonists, comprising administering to the patient an effective amount of the composition of claim 11.

19) A human growth hormone receptor antagonist, comprising:
(a) human growth hormone receptor antagonist G120K, wherein at least one amino acid of human growth hormone receptor antagonist G120K has been mutated to cysteine, and wherein the amino acid mutated to cysteine is T3, E39, P48, Q69, N99, G120, T142, H151 or combinations thereof; and
(b) a polytethylene glycol molecule conjugated to each cysteine residue in the human growth hormone receptor antagonist G120K mutant, wherein the polytethylene glycol molecule is a linear structure or a branched structure, wherein the polyethylene glycol molecule contains a malemide group for conjugation to a free sulfhydryl group, and wherein the polyethylene glycol molecule is selected from the group consisting of a 20 kDa linear molecule, a 40 kDa linear molecule, and a 40 kDa branched molecule.

20) The composition of claim 19,
(a) wherein the human growth hormone receptor antagonist is encoded by a DNA sequence having at least 95% identity to a DNA sequence selected from the group consisting of SEQ ID NOs: 5, 7, 9, 11, 13, 15, 17, 19, 21, and 23; and
(b) wherein the human growth hormone receptor antagonist has an amino acid sequence selected from the group consisting of SEQ ID NOs: 6, 8, 10, 12, 14, 16, 18, 20, 22, and 24.


SEQUENCE LISTING (24 sequences)

SEQ ID NO: 1 - DNA, 645 bp, Artificial Sequence
  WThGH (DNA) Human Growth Hormone
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 2 - protein, 214 aa, Artificial Sequence
  WThGH (amino acid) Human Growth Hormone
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 3 - DNA, 645 bp, Artificial Sequence
  G120K (DNA) Human Growth Hormone Antagonist G120K
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 4 - protein, 214 aa, Artificial Sequence
  G120K (amino acid) Human Growth Hormone Antagonist G120K
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 5 - DNA, 645 bp, Artificial Sequence
  G120K-T3C (DNA) Synthetic Constructs / Mutant Human Growth Hormone Antagonists
atggcgcatcaccaccaccatcacggcagcagcggcgagaacctgtacttccagggtggaagctcgggattcccatgcattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 6 - protein, 214 aa, Artificial Sequence
  G120K-T3C (amino acid) Synthetic Constructs / Mutant Human Growth Hormone Antagonists
MAHHHHHHGSSGENLYFQGGSSGFPCIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 7 - DNA, 645 bp, Artificial Sequence
  G120K-E39C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaagtgccagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 8 - protein, 214 aa, Artificial Sequence
  G120K-E39C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKCQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 9 - DNA, 645 bp, Artificial Sequence
  G120K-P48C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaactgtcagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 10 - protein, 214 aa, Artificial Sequence
  G120K-P48C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNCQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 11 - DNA, 645 bp, Artificial Sequence
  G120K-Q69C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacagtgcaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 12 - protein, 214 aa, Artificial Sequence
  G120K-Q69C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNPQTSLCFSESIPTPSNREETQCKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 13 - DNA, 645 bp, Artificial Sequence
  G120K-N99C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgcctgcagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 14 - protein, 214 aa, Artificial Sequence
  G120K-N99C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFACSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 15 - DNA, 645 bp, Artificial Sequence
  G120K-T142C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 16 - protein, 214 aa, Artificial Sequence
  G120K-T142C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 17 - DNA, 645 bp, Artificial Sequence
  G120K-H151C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcatgcaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 18 - protein, 214 aa, Artificial Sequence
  G120K-H151C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSCNDDALLKNYGLLYCFRKDMDKVETFLRIVQCRSVEGSCGF

SEQ ID NO: 19 - DNA, 645 bp, Artificial Sequence
  G120K-N99C-dPEGX-H151C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgcctgcagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcatgcaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 20 - protein, 214 aa, Artificial Sequence
  G120K-N99C-dPEGX-H151C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFACSLVYGASDSNVYDLLKDLEEKIQTLMGRLEDGSPRTGQIFKQTYSKFDTNSCNDDALLKNYGLLYCFRKDMDKVETFLRIVQCRSVEGSCGF

SEQ ID NO: 21 - DNA, 645 bp, Artificial Sequence
  G120K-T142C-dPEGX-N99C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgcctgcagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 22 - protein, 214 aa, Artificial Sequence
  G120K-T142C-dPEGX-N99C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFACSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 23 - DNA, 645 bp, Artificial Sequence
  G120K-T142C-dPEGX-H151C (DNA) Synthetic Constructs / Mutant Human Growth Hormone Antagonists
atggcgcatcaccaccaccatcacggcagcagcggcgagaacctgtacttccagggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcatgcaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 24 - protein, 214 aa, Artificial Sequence
  G120K-T142C-dPEGX-H151C (amino acid) Synthetic Constructs / Mutant Human Growth Hormone Antagonists
MAHHHHHHGSSGENLYFQGGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSCNDDALLKNYGLLYCFRKDMDKVETFLRIVQCRSVEGSCGF